(19) United States
(12) Patent Application Publication
(10) Pub. No.: US 2001/0037811 A1
(43) Pub. Date: Nov. 8, 2001

(54) Method and apparatus for arthritis diagnosis

(76) Inventors: Juergen Beuthan, Berlin (DE); Peter Mayer, Muenchen (DE); Georg Metzger, Hagenau (DE); Monika Reuss-Borst, Bad Kissingen (DE); Helmut Rost, Uttenreuth (DE); Alexander Scheel, Gottingen (DE); Volker Tresp, Muenchen (DE)

Correspondence Address: SCHIFF HARDIN & WAITE, 6600 SEARS TOWER, 233 S WACKER DR, CHICAGO, IL, 60606-6473, US

(21) Appl. No.: 09/775,070

(22) Filed: Feb. 1, 2001

(30) Foreign Application Priority Data
Feb. 4, 2000 (DE) 10004989.3

(51) Int. Cl. A61B 19/00

(52) U.S. Cl. 128/897

(57) ABSTRACT

In a method and apparatus for determining the circumference of a finger or toe joint, in particular a proximal interphalangeal joint, to be evaluated in the context of an arthritis examination of an examination subject, the joint is irradiated using a light source and at least one two-dimensional projection image is recorded using a camera apparatus. From the projection image, the diameter of the joint is determined by means of an automatic edge detection method, and the circumference is calculated on the basis of the diameter. Additionally, a diaphanoscopic examination of the joint can be made with the same apparatus, and the results combined with the diameter information to identify or monitor a degree or progress of the inflamation.

### BACKGROUND OF THE INVENTION

1. Field of the Invention

The present invention is directed to a method for determining the circumference of a digit joint (finger joint or toe joint,) in particular a proximal interphalangeal joint, to be evaluated in the context of an arthritis examination of an examination subject.

2. Description of the Prior Art

In the field of medicine, the task increasingly arises of being able to detect and evaluate pathological changes in tissue caused by metabolism in a simple manner that disturbs the patient as little as possible. An example of such pathological tissue changes is represented by rheumatic joint changes or rheumatic diseases in the area of the tissue. The finger joints, and above all the proximal interphalangeal joints, are particularly affected by such changes. Chronic arthritis often occurs as a symptom of aging, but also can be observed in younger people. The rheumatoid inflammation process of synovialis, i.e., an inflammation in the region of the joint, is associated with a swelling in the joint region. The stronger the degree of inflammation and thus of the rheumatic attack, the stronger is the swelling and thus the greater the circumference at the joint. This characteristic value describing the state of the joint is recorded in the context of an arthritis examination for diagnostic purposes, but the determination of the circumference is made manually by means of a flexible measuring strip that is placed around the joint. The measurement value is read from the measuring strip by the rheumatologist or by a medical technician, and is allocated to the respective finger joint and entered into the patient's database. The measurement precision is thereby at least +/−0.5 mm, but has proven to be larger in practice, which is related to the fact that the measuring strip can be drawn around or placed on the joint with varying strength, because the tissue and the skin are compressible. Reliable measurement values, which in particular could be used in the context of a running diagnosis, thus are not obtained.

### SUMMARY OF THE INVENTION

An object of the present invention is to provide a method which allows the circumference of a digit joint, which represents an important characteristic value for the diagnosis of arthritis, to be measured with sufficient precision.

This object is achieved in accordance with the inventive method by irradiating the joint using a light source, and receiving at least one two-dimensional projection image using a camera apparatus, with the diameter of the joint in the projection image the being determined using an automatic edge detection method, and wherein the circumference is calculated on the basis of the joint diameter.

The inventive method enables a contact-free measurement of the circumference, which can be determined very precisely by means of the automatic edge detection method that is carried out at a computer. In an embodiment of the invention, the joint is irradiated, using a light source that is moved around the joint along a circular path, at various angular positions at a particular angular spacings. For each irradiation position the two-dimensional projection image is recorded using the camera apparatus. For each projection image the diameter (dₙ) of the joint is determined using the automatic edge detection method and, on the basis of the diameter, the circumference is determined according to the formula

$$U = {\sum }_{n = 1}^{n = x} {d}_{n} arc Δϕ$$

where U=circumference

x=number of projection images

dₙ=joint diameter in the respective projection image

Δφ=angular difference between two angular positions.

In this embodiment of the method, the circumference is determined in contact-free fashion by recording a multiplicity of two-dimensional projection images of the joint illuminated from behind. Each recorded projection image, in which the joint is visible as a quasi-shadow, is evaluated using an automatic edge detection method, and the joint diameter in each projection is determined. In the context of the edge detection method, the diameter of the joint in the respective projection exposure should always be determined at the same location in relation to the longitudinal axis of the digit, so that it is ensured that the computer-calculated circumference is the circumference at a particular point of the joint. In the context of a running monitoring, it should be ensured that in subsequent determinations of circumference, the circumference is always determined at the same location. Edge detection methods are sufficiently known, with which it is possible to determine the transition between the skin and the air with the greatest precision. The projection images are recorded at various angular positions, the light source being moved about the joint along the circular path for this purpose. The determination of the circumference is made from the sum of the differential radian measures, according to the indicated equation. In this way, the circumference of the joint can be determined with particularly advantageously high precision. On the basis of the change of the circumference overtime, the degree of inflammation therefore can be diagnosed in a diagnostically effective manner. If the circumference increases, the degree of inflammation has increased, whereas if the joint swelling decreases, this indicates an improvement or a therapeutic success.

The projection images should be recorded over an angular distance of at least 180°, in order to ensure that the joint has been completely scanned. The angular difference Δφ should be selected less than 5°, in particular less than 30, and an angular difference of 2° is preferred. This ensures a sufficiently high number of recorded projection images. The higher the number, the more precisely the circumference can be determined. Given a step-by-step scanning with an angular difference of 2°, a total of 90 projection images are recorded, permitting a sufficiently precise determination.

The circumference, determinable with a high degree of precision by means of the inventive method, gives the diagnosing physician a characteristic value that is of excellent utility for the subsequent diagnosis and therapeutic monitoring.

In addition to a method for determining the characteristic value, the invention relates to a method for determining one or more information values of a digit joint, in particular of a proximal interphalangeal joint, of a subject that are relevant for a subsequent arthritis diagnosis,. This method combines the determination specified above of the circumference of the joint—forming a first characteristic value—with the execution of a diaphanoscopic examination of the joint, in which the joint is transilluminated with light having a wavelength in the region of the optical tissue window, and a scattered light distribution is recorded in the form of a spread function, after which one or more characteristic values, that are characteristic for the properties of the spread function, are determined by computer based on the curve of the spread function. Subsequently, the determined circumference and function characteristic values are combined with one another by computer, in order to determine one or more information emitted as outputs, on the basis of which the physician obtains important indications relating to the state of the joint that are useful for diagnosis and therapy. A diaphanoscopic examination method of this sort is specified in detail in WO 99/04683, corresponding to co-pending U.S. application Ser. No. 09/463,110 filed Jan. 19, 2000 (“Method for Evaluating a Distribution of Scattered Light, Obtained by Local Transirradiation of a living Organism by Determining Characteristic Values, Abraham-Fuchs et al.). The teachings of this co-pending United States application are incorporated herein by reference.

On the basis of the diaphanoscopic examination, a series of characteristic values that permit a description of the state of the tissue are provided to the physician. These characteristic values are determined from the scattered light distribution. For this purpose, the joint is transilluminated and, using a camera apparatus, the transmitted scattered light exiting at the opposite side is recorded and evaluated. The scattered light distribution is dependent on the state of the joint, which becomes worse as the inflammation process increases, with the result that the transmission characteristic becomes worse due to a cloudiness resulting from inflammation hypertrophy, resulting in a changing scattered light distribution. The results of this change can in turn be seen in the characteristic values that can be obtained. On the basis of these values, the rheumatologist obtains important information. According to the invention, in addition to this diaphanoscopic examination, the circumference also is determined, which likewise forms an important characteristic value, and, using a computer, the characteristic values obtained from the different methods and combined with one another in order to determine the information values. A neural network model preferably is used for the computer-supported combination and in order to determine the information values. Alternatively to a neural network model, other networks, models or logics may of course also be used that enable the computer-aided combination and determination of information values. The information values based on the various characteristic values are even more important in their diagnostic content, and enable the physician to make a still better diagnosis.

In addition, the invention relates to an apparatus that is suitable for the execution of one or both of the above-described basic embodiments of the inventive methods. The apparatus includes a finger or toe support on which the finger or toe having the joint to be examined can be fixed, at least one light source with which the joint is irradiated, a camera apparatus for the recording of irradiation images, and a computer for processing and evaluation of the irradiation images supplied thereto.

In am embodiment of the inventive apparatus, the light source or sources and a camera apparatus located on the other side of the joint can be moved around the joint along a circular path.

The inventive apparatus is fashioned for the execution either the embodiment of the method for determining the circumference or for executing embodiment of the method requiring both a determination of circumference and a diaphanoscopic examination. The computer must merely be correspondingly designed—in the one case, for the calculation of the circumference, and in the other case additionally for the evaluation of the scattered light distributions. The two-dimensional projection images for determining the circumference, as well as the scattered light distributions, are recorded using the camera apparatus, which can be moved together with the light source and which is arranged on the opposite side—i.e., the side facing away from the light source—of the finger or joint. For recording the two-dimensional projection images, a first light source for irradiation of the joint is used, preferably a flat light source, since the joint must be illuminated from the rear only so that the projection image can be recorded. For carrying out the diaphanoscopic examination, and thus for the recording of the scattered light distributions, it is preferable to use a second light source, preferably a laser light source, for punctiform irradiation of the joint. This light source is preferably arranged close to the first light source so as to be movable therewith, but it is also possible to arrange this second light source immovably, since a transillumination of the joint always takes place from the same side of the joint. In the simplest realization, the light source required in the context of the measurement of the circumference is stationary, as is the camera apparatus. Here a projection image is recorded in only one position, and the circumference is calculated on the basis of this one diameter. This very simple realization is possible in particular for home application, for rapid monitoring by the patient himself or herself. A diaphanoscopic examination is not absolutely required here.

According to a further embodiment of the invention, the camera apparatus can be arranged opposite the (first (and second)) light source. In this case, the light—regardless of whether a projection image or scattered light is being recorded—enters directly into the camera apparatus and is supplied to this apparatus to the computer, connected downstream. As an alternative, it is possible to employ a deflecting mirror, arranged opposite the light source and movable therewith. This mirror deflects the incident radiation by a 90° angle onto the camera apparatus arranged adjacent thereto, the camera apparatus also being moved with the light source and mirror. This specific embodiment is advantageous in particular from a structural point of view, since in this way a hindrance of a rotational motion due to the camera apparatus, which is located relatively far away if positioned opposite, is avoided; this camera apparatus may otherwise collide with the adjacent fingers, and in such cases the required rotational motion is not always ensured.

The light source (or two light sources), arranged in the interior of an essentially hollow cylindrical mount, can be moved via a drive mechanism. The light source(s), the camera apparatus, and, if necessary, the deflecting mirror, are provided on the interior wall of the mount, which is open at one side and preferably closed at the opposite side. The mount is open at only one side in order to enable introduction of the digit, such as a finger. The darkness prevailing in the interior enables the recording of radiation images of sufficiently good quality, not adversely affected by parasitic radiation. The drive is preferably a stepper motor that, via a driven worm, engages a worm gear arranged on the mount. By means of this drive mechanism the mount can be rotated step-by-step into predetermined angular positions in which it is stopped and remains until advanced again. By this means, in a simple manner a step-by-step rotation of the mount and therefore of the recording components is enabled. The control of the mount drive mechanism should be designed so that the angular differences can be selected freely and the drive mechanism rotates the mount correspondingly.

During the motion of the mount around the joint, the first light source can emit light continuously, whereby at the camera apparatus images are recorded only at the selected angular positions. As an alternative, a pulsed operation of the light source, correlated with the stopping of the mount in the angular positions, can be employed. The recording of the projection images and of the scattered light distributions can take place one after the other, or, alternatively, it is also possible to begin with the recording of the projection images and, when the angular position has been reached in which the scattered light distributions are recorded (as a rule, this is the position in which the second light source is located in perpendicular position under the joint), to record the scattered light distributions, after which the recording of the projection images is continued.

Since the inventive apparatus is intended to make it possible to be able to measure the finger or toe joints of all fingers/toes of a living subject, care must be taken that, given digits of different lengths, the joint to be examined can always be brought into the image recording region. For this purpose, according to the invention the finger or toe support housed in the interior of the mount can be moved in the direction of its longitudinal axis, a stepper motor likewise preferably being used for this purpose. This version of the invention makes it possible to fix an arbitrary finger of a hand on the finger or toe support, and subsequently to position it precisely. The use of a movable support additionally has the advantage that it is possible to work with a single laser light source in the context of the carrying out of the diaphanoscopic examination. In the context of this examination, as is specified in detail in U.S. application Ser. No. 09/463,110, the joint is irradiated with the laser light at a multiplicity of locations disposed in a direction perpendicular to the joint gap, i.e., a number of scattered light distributions are recorded and subsequently evaluated. The possibility of moving the finger or toe support makes it possible to move this support past the laser light source, which may be a laser diode, in step-by-step fashion, and thus to move the joint past the laser light source in order to scan it.

A further advantage of this embodiment is that the optimal locus of radiation for the diaphanoscopic examination can be found in an equally simple manner. The above-cited number of irradiation points are placed around an optimal locus of radiation. The determination of the optimal locus of radiation takes place according to the method as specified in application Ser. No. 09/463,110. In order to find this locus, it is also required that the joint be transilluminated at a number of points. If the support can be moved, the joint can also be moved past the stationary laser light source for this purpose.

A common control unit for controlling the operation of the light source(s) and for the movement of the mount, as well as the movement of the finger or toe support, is preferably provided.

### DESCRIPTION OF THE DRAWINGS

FIG. 1 is a schematic illustration of an inventive apparatus.

FIG. 2 is a schematic illustration for the representation of a cross-section of a finger joint for explaining the calculation of the circumference.

FIG. 3 is a side view of a calibration bar for calibrating the apparatus according to FIG. 1.

### DESCRIPTION OF THE PREFERRED EMBODIMENTS

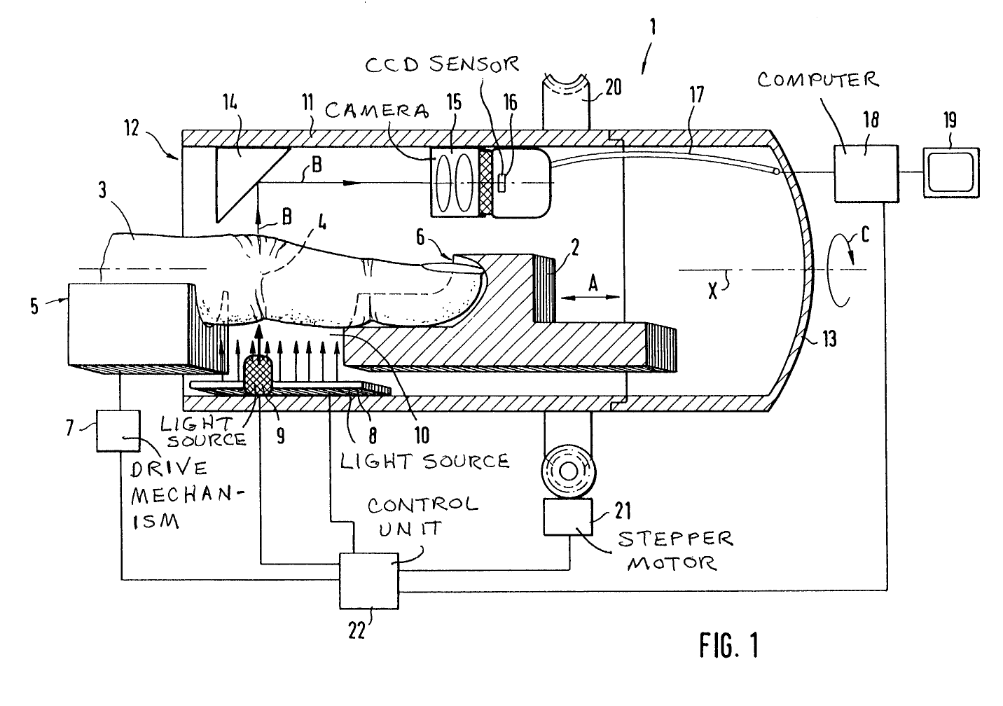

FIG. 1 shows, in the form of a schematic drawing, an inventive apparatus 1 for carrying out both versions of the inventive method in the context of a finger examination. The invention, however, can also be used to conduct the same examination for a toe. The apparatus 1 has a finger support 2, on which a finger 3 whose joint4 (a proximal interphalangeal joint) is to be examined can be fastened using fixing means not shown in more detail. The support 2, shown in a partial section, has a finger trough 5 in which the finger rests. The fingertip is held in a recess 6 that prevents the finger from being lifted accidentally. The finger support 2 can be moved back and forth in the direction of the double arrow A via a drive mechanism 7, in order to move and adjust the joint 4 in relation to light sources 8, 9. The light sources 8, 9 are used for irradiation of the joint 4. In order to enable this, an opening 10 is provided on the finger support 2, irradiation taking place through this recess. The opening is, for example, 30 mm long.

The finger support 2 protrudes into a hollow cylindrical mount 11, which has an entry opening 12 on its front side and has a closing wall 13 on its back side. In this way it is ensured that it is sufficiently dark in the interior of the mount 11 so that the radiation images to be exposed will not be falsified by incident light.

The two light sources 8, 9 are arranged on the inner wall of the mount 11. On the opposite wall there is arranged a deflecting mirror 14, by means of which incident radiation from the finger joint 4 is deflected onto a camera apparatus 15, which is a miniature camera module having a CCD sensor 16 (see arrows B). The CCD sensor 16 can have a resolution of 640×480 pixels (=350,000 pixels). Due to its integration in the interior of the mount 11, the camera apparatus 15 itself should be as small as possible, so that the apparatus as a whole can be designed to be small. The image signals read out by the CCD sensor 16 are given, via a line connection 17, to a computer 18, to which a display 19 in the form of monitor is connected downstream.

The light sources 8, 9, the deflecting mirror 14, and the camera apparatus 15 are fixed on the inner wall of the mount 11. The mount 11 itself can be moved around the axis of rotation X via a worm gear 20 (this can also be a toothed wheel or the like) and a stepper motor 21 that engages this gear 20 (see arrow C). If the mount 11 is rotated, the light sources 8, 9, the deflecting mirror 14, and the camera apparatus 15 are moved around the finger joint 4. In this way, it is possible to irradiate the finger joint 4 with light from the first light source 8, which is a flat light source, from a number of positions, and to record different projection images using the camera apparatus 15. The operation of the light sources 8, 9 and of the stepper motors 7 and 21 is controlled via a central control unit 22.

Using the inventive apparatus, two-dimensional projection image exposures of the joint 4 are possible. For this purpose, the mount is moved into an initial position, for example the position shown in FIG. 1, in which the light source 8 is located in perpendicular position under the joint 4. Using the stepper motor 21, the mount 11 is now rotated step-by-step through 180°. At angular positions respectively spaced 2° from one another, a projection image exposure is respectively recorded using the camera apparatus 15. For this purpose, the light source 8 is switched on in this position, or, as an alternative, it can also be operated during the entire 180° rotation. The geometrical two-dimensional projection of the joint 4 is deflected via the deflecting mirror 14 onto the camera apparatus 15, which records the image in the CCD sensor 16. This image is subsequently read out into the computer 18. Given an angular difference between the recording positions of Δφ=20°, with a rotation scan of (φ=180° a total of 90 projection images result, in each of which the finger joint is shown in a slightly different position.

Using an edge detection method, the computer 18 is now able to determine the diameter of the finger joint shown in each projection, so that overall, given a number x of projection images, a corresponding number of diameters dₙ is obtained, which are respectively allocated to different angular positions. On the basis of these determined diameter values, the computer 18 can now acquire the precise circumference of the finger joint 4 on the basis of the equation

$$U = {\sum }_{n = 1}^{n = x} {d}_{n} arc Δϕ$$

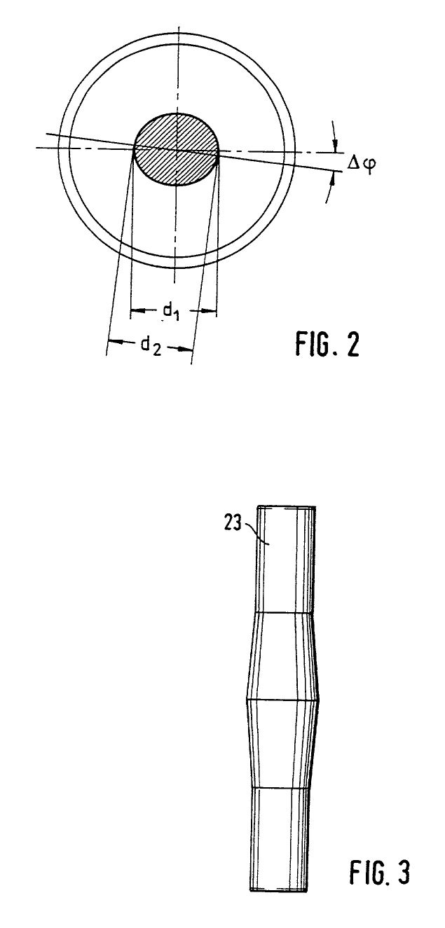

On the basis of two diameters d₁ and d₂, FIG. 2 illustrates how the diameter changes as the recording position changes, and how the above equation results. The circumference of the finger joint 4 increases as the degree of inflammation advances. Because the apparatus 1 determines the diameter without contact, a high precision is possible in the determination of the circumference, and comparable values can be recorded in the context of running tests in order to observe the development of the inflammation. The circumference is a useful characteristic value for the rheumatologist's diagnosis.

Besides this embodiment for determining circumference, using the apparatus 1 a number of scattered light distributions also can be recorded and evaluated, in order to determine further characteristic values that are important for the subsequent diagnosis. In the context of the diaphanoscopic examination, the finger joint is transilluminated with light from the light source 9. In the depicted exemplary embodiment, this is a single laser diode that emits narrowband light having a wavelength in the region of the optical tissue window. This light penetrates the joint 4, which is transparent in the region of the joint gap as well as in the cartilage region and in the region of the joint fluid, and exits at the opposite side of the joint 4 as scattered light. This scattered light is deflected via the deflecting mirror 14 into the camera apparatus 15, where the scattered light distribution is recorded and is subsequently read out to the computer 18. In order to carry out this diaphanoscopic examination, the finger joint 4 first must be positioned over the light source 9 in such a way that the optimal locus of radiation at the joint 4 is located precisely over the light source 9. In order to determine the optimal locus of radiation, the region of the joint 4 in which the optimal examination locus is probably located is first positioned roughly over the light source 9, and subsequently transilluminations are made sequentially at various points located next to one another, in order to record first scattered light distributions in the form of locus-related spread functions, in particular point spread functions, which are subsequently evaluated at the computer 18. The precise procedure for the determination of this optimal locus of examination is specified in application Ser. No. 09/463,110. For the sequential illumination, the finger support 2 is displaced instep-by-step fashion in relation to the light source 9, which takes place by means of the stepper motor 7.

Once the optimal locus of examination has been determined by the computer 18, the finger support 2 is positioned correspondingly, so that this optimal locus of examination is located precisely over the light source 9. The computer 18 forwards the corresponding information to the control units 22, which correspondingly controls the stepper motor 7. It should be noted that this control unit 22 can of course be integrated into the computer 18, that is, the computer means 18 directly controls the aforementioned components.

After successful positioning, the actual diaphanoscopic examination takes place, in the course of which a number of scattered light distribution images are recorded at various positions around the optimal locus of examination. For this purpose, the finger support 2 is again displaced in step-by-step fashion, the individual loci of radiation being, for example, respectively spaced 200 μm from one another, symmetrical to the center of the joint gap. At each location, the light source 9 is operated briefly in order to record the scattered light distribution. The individual scattered light distributions are evaluated at the computing unit 18 for the determination of one or more distribution-related characteristic values. An evaluation method is specified in application Ser. No. 09/463,110.

It is possible to display the individual characteristic values themselves to the physician on the display 19. It is useful, however, to combine the characteristic values resulting from the determination of circumference and the scattered light distribution analysis with one another, because, due to their dependence on the degree of inflammation, they all contain information describing this degree of inflammation indirectly. If these characteristic values describing the state of the joint are combined with one another, information values that are still more effective for diagnostic purposes can be determined. For this purpose, the characteristic values are processed at the computer 18 in a neural network.

FIG. 3 shows a calibration bar 23 that is non-reflecting and that has in its middle region a thickening in the shape of a truncated cone that is for example 20 mm in diameter. For calibration purposes, this thickening is moved into the illumination region and the calibration measurements are carried out.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for determining a circumference of a digit joint comprising the steps of:
irradiating said joint with a light source, and thereby producing light attenuated by said joint;
recording at least one two-dimensional projection image from said attenuated light using a camera;
determining a diameter of said joint from said projection image using an automatic edge detection method; and
calculating the circumference of the joint from said diameter.

2. A method as claimed in claim 1 wherein the steps of irradiating said joint and recording at least one two-dimensional projection image comprise moving said light source around said joint along a circular path through a plurality of angular positions spaced from each other by an angle difference and, from each angular position, recording said two-dimensional projection image, thereby obtaining a number of two-dimensional projection images, determining the diameter of the joint using said automatic edge detection method in each of said two-dimensional projection images, and wherein the step of calculating the circumference comprises calculating the circumference of said joint according to:
$$U = {\sum }_{n = 1}^{n = x} {d}_{n} arc Δϕ$$
wherein U is the circumference, x is said number of projection images, dₙ is the joint diameter in the respective projection images, and Δφ is said angle difference.

3. A method as claimed in claim 2 wherein the step of rotating said light source comprises rotating said light source through an angular path of at least 180°.

4. A method as claimed in claim 2 wherein Δφ is less than 5°.

5. A method as claimed in claim 2 wherein Δφ is less than 3°.

6. A method as claimed in claim 2 wherein Δφ is 2°.

7. A method as claimed in claim 1 comprising the additional steps of:
conducting a diaphanoscopic examination of said joint by transilluminating said joint with light having a wavelength in a region of an optical tissue window associated with said joint, and thereby producing scattered light from said joint;
recording a scattered light distribution of said scattered light as a spread function, said spread function having a curve associated therewith;
in a computer, identifying at least characteristic value representing a characteristic property of said spread function by analyzing said curve of said spread function in said computer; and
in said computer, combining said characteristic value and said diameter to obtain information representing a degree of inflamation of said joint.

8. A method as claimed in claim 7 wherein the step of combining said characteristic value and said diameter comprises supplying said characteristic value and said diameter to a neural network to obtain said information as an output of said neural network.

9. An apparatus for determining a circumference of a digit joint, comprising:
a digit support adapted to receive a digit, having a joint therein, of an examination subject in a fixed position;
a light source disposed for irradiating said joint in said finger support with light, to obtain attenuated light attenuated by said joint;
a camera disposed so that said attenuated light is incident thereon for recording at least one two-dimensional projection image of said joint from said attenuated light, said camera emitting output signals representing said projection image; and
a computer supplied with said signals from said camera for determining a diameter of said joint in said projection image by subjecting said projection image to an automatic edge detection method, and for calculating a circumference of said joint from said diameter.

10. An apparatus as claimed in claim 9 further comprising a rotational apparatus on which said light source and said camera are mounted for rotating said light source and said camera around said joint to irradiate said joint with light from said light source from a plurality of different angular positions, and wherein said camera records said projection image from each of said angular positions, to obtain a plurality of projection images, and wherein said computer is supplied with signals from said camera for each of said plurality of said projection images and determines a diameter in each of said projection images using said automatic edge detection method, and calculates said circumference from the respective diameters in the plurality of projection images.

11. An apparatus as claimed in claim 10 wherein said camera and said light source are mounted opposite each other in said rotational apparatus.

12. An apparatus as claimed in claim 10 further comprising a deflecting mirror mounted in said rotational apparatus opposite said light source, on which said attenuated light is incident, said deflecting mirror deflecting said attenuated light by a 90° angle, and wherein said camera is mounted in said rotational apparatus relative to said deflecting mirror so that said attenuated light deflected by said 90° angle is incident on said camera.

13. An apparatus as claimed in claim 12 wherein said rotational apparatus comprises a hollow-cylindrical housing having an interior in which said light source, said camera and said deflecting mirror are mounted, and a drive mechanism for rotating said housing.

14. An apparatus as claimed in claim 13 wherein said drive mechanism comprises a stepper motor with a transmission mechanism producing a driving engagement between said stepper motor and said housing.

15. An apparatus as claimed in claim 14 wherein said stepper motor rotates said housing in a plurality of successive steps respectively corresponding to said angular positions, and stops and holds said housing at each of said angular positions while one of said projection images is recorded by said camera.

16. An apparatus as claimed in claim 15 wherein said light source emits light continuously as said housing is rotated by said stepper motor.

17. An apparatus as claimed in claim 15 wherein said light source is operated to emit light in pulses correlated with said angular positions.

18. An apparatus as claimed in claim 14 wherein said housing has a longitudinal axis around which said housing rotates, and wherein said digit support is movable in said interior of said housing along said longitudinal axis.

19. An apparatus as claimed in claim 18 comprising a common control unit connected to said light source, said housing and said finger support, for coordinating operation of said light source, operation of said stepper motor, and movement of said finger support relative to each other.

20. An apparatus as claimed in claim 9 wherein said light source is a substantially planar light source.

21. An apparatus as claimed in claim 20 wherein said substantially planar light source comprises a plurality of laser diodes arranged in a plane.

22. An apparatus as claimed in claim 9 wherein said camera is disposed opposite said light source.

23. An apparatus as claimed in claim 9 further comprising a deflecting mirror disposed opposite said light source with said attenuated light incident on said deflecting mirror, said deflecting mirror deflecting said attenuated light by a 90° angle, and said camera being disposed relative to said deflecting mirror so that said attenuated light deflected by said 90° angle is incident on said camera.

24. An apparatus for examining a digit joint comprising:
a digit support adapted to receive a digit, containing a joint, thereon in a fixed manner;
a first light source for irradiating said joint in said digit support with light to produce attenuated light attenuated by said joint;
a second light source for irradiating said joint with light having a wavelength in an optical tissue window associated with said joint to produce scattered light from said joint;
a camera on which said attenuated light and said scattered light are incident for recording a two-dimensional projection image of said joint from said attenuated light and for recording a scattered light distribution from said scattered light, said camera producing first signals representing said projection image and second signals representing said scattered light distribution; and
a computer supplied with said first signals and said second signals for, from said first signals, determining a diameter of said joint in said projection image using an automatic edge detection method and for calculating a circumference of said joint from said diameter, and for identifying a spread function curve, from said second signals, associated with said scattered light distribution and for identifying a characteristic value of said spread function curve, and for combining said circumference and said characteristic value to produce information representing a degree of inflamation of said joint.

25. An apparatus as claimed in claim 24 further comprising a rotational apparatus on which said first and second light sources and said camera are mounted for rotating said first and second light sources and said camera around said joint to irradiate said joint with light at least from said first light source from a plurality of different angular positions, and wherein said camera records said projection image from each of said angular positions, to obtain a plurality of projection images, and wherein said computer is supplied with signals from said camera for each of said plurality of said projection images and determines a diameter in each of said projection images using said automatic edge detection method, and calculates said circumference from the respective diameters in the plurality of projection images.

26. An apparatus as claimed in claim 25 wherein said camera and said first and second light sources are mounted opposite each other in said rotational apparatus.

27. An apparatus as claimed in claim 25 further comprising a deflecting mirror mounted in said rotational apparatus opposite said first and second light sources, on which said attenuated light and said scatter light are incident, said deflecting mirror deflecting said attenuated light and said scattered light by a 90° angle, and wherein said camera is mounted in said rotational apparatus relative to said deflecting mirror so that said attenuated light and said scattered light deflected by said 90° angle are incident on said camera.

28. An apparatus as claimed in claim 27 wherein said rotational apparatus comprises a hollow-cylindrical housing having an interior in which said first and second light sources, said camera and said deflecting mirror are mounted, and a drive mechanism for rotating said housing.

29. An apparatus as claimed in claim 27 wherein said drive mechanism comprises a stepper motor with a transmission mechanism producing a driving engagement between said stepper motor and said housing.

30. An apparatus as claimed in claim 29 wherein said stepper motor rotates said housing in a plurality of successive steps respectively corresponding to said angular positions, and stops and holds said housing at each of said angular positions while one of said projection images is recorded by said camera.

31. An apparatus as claimed in claim 30 wherein at least said first light source emits light continuously as said housing is rotated by said stepper motor.

32. An apparatus as claimed in claim 30 wherein at least said first light source is operated to emit light in pulses correlated with said angular positions.

33. An apparatus as claimed in claim 28 wherein said housing has a longitudinal axis around which said housing rotates, and wherein said digit support is movable in said interior of said housing along said longitudinal axis.

34. An apparatus as claimed in claim 33 comprising a common control unit connected to said light source, said housing and said finger support, for coordinating operation of said light source, operation of said stepper motor, and movement of said finger support relative to each other.

35. An apparatus as claimed in claim 24 wherein said first light source is a substantially planar light source.

36. An apparatus as claimed in claim 35 wherein said substantially planar light source comprises a plurality of laser diodes arranged in a plane.

37. An apparatus as claimed in claim 24 wherein said camera is disposed opposite said first and second light sources.

38. An apparatus as claimed in claim 9 further comprising a deflecting mirror disposed opposite said first and second light sources with said attenuated light and said scattered light incident on said deflecting mirror, said deflecting mirror deflecting said attenuated light and said scattered light by a 90° angle, and said camera being disposed relative to said deflecting mirror so that said attenuated light and said scattered light deflected by said 90° angle is incident on said camera.

39. An apparatus as claimed in claim 24 wherein said second light source is a laser diode.
